(19) United States
(12) Patent Application Publication
(10) Pub. No.: US 2006/0070880 A1
(43) Pub. Date: Apr. 6, 2006

(54) Methods and apparatus for manipulating separation media

(75) Inventors: Johan R. Goudberg, San Jose, CA (US); James C. Burrows, Burlingame, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/215,935

(22) Filed: Aug. 31, 2005

Related U.S. Application Data
(60) Provisional application No. 60/606,537, filed on Aug. 31, 2004.

(51) Int. Cl. C07K 1/26; G01N 27/447

(52) U.S. Cl. 204/450; 204/603

(57) ABSTRACT

Methods and apparatus for manipulating separation media in the context of filling one or more capillaries with a separation medium for electrophoresis. A polymer-displacement pump system and method for reciprocating a pump piston in a first direction to draw fresh fluid into a chamber, and reciprocating the pump piston in a second direction to cause the fresh fluid to exit the chamber and fill a single capillary or multi-capillary array. The pump piston movement can be electrically controlled.

### PRIORITY APPLICATION

This application claims the benefit of U.S. Provisional Patent Application No. 60/606,537, filed Aug. 31, 2004, the entire disclosure of which is hereby incorporated by reference as if set forth fully herein.

### FIELD

The present teachings relate to methods and apparatus for manipulating separation media, for example, in the context of filling one or more capillaries with a separation medium for electrophoresis.

### INTRODUCTION

Prior capillary electrophoresis systems have employed syringes for filling elongated capillaries with separation media (e.g., flowable polymer). User skill and time are required to set up most syringe systems. Also, with most syringe systems, there is a significant amount of manual intervention needed when priming the system.

### SUMMARY

According to various embodiments, a polymer-displacement pump system can be provided as can a method. The method can involve reciprocating a pump piston in a first direction to draw fresh fluid into a chamber, and reciprocating the pump piston in a second direction to cause the fresh fluid to exit the chamber and fill a single capillary or multi-capillary array. The pump piston movement can be electrically controlled.

According to various embodiments, a pump system is provided that can comprise at least one first block and an outlet opening formed in the at least one first block, a fluid chamber formed in the at least one first block and in fluid communication with the at least one outlet opening, a buffer storage container connector adapted to retain a buffer storage container in fluid communication with the fluid chamber, an electrode adjacent the buffer storage container connector and adapted to be in electrical communication with liquid in a buffer storage container when a buffer storage container is connected to the buffer storage container connector, a polymer container connector adapted to form a fluid communication with polymer in a polymer container, and a reciprocating piston pump in fluid communication with the fluid chamber and the polymer container connector.

According to various embodiments, a capillary electrophoresis system is provided that can comprise: a pump system comprising a first block and an outlet opening formed in the first block, a fluid chamber formed in the first block and in fluid communication with the at least one outlet opening, a buffer storage container connector adapted to retain a buffer storage container in fluid communication with the fluid chamber, an electrode adjacent the buffer storage container connector and adapted to be in electrical communication with liquid in a buffer storage container when a buffer storage container is connected to the buffer storage container connector, a polymer container connector adapted to retain a polymer container, and a reciprocating piston pump in fluid communication with the fluid chamber and the polymer container connector; a temperature regulated chamber comprising a container portion and an opening; a single capillary or multi-capillary array disposed within the container portion and coupled to the outlet through the opening; an excitation source; and a detector assembly coupled to the single capillary or multi-capillary array.

According to various embodiments, a method is provided that can comprise reciprocating a pump piston in a first direction to draw fresh fluid into a chamber, and reciprocating the pump piston in a second direction to cause the fresh fluid to exit the chamber and fill a single capillary or multi-capillary array of a capillary electrophoretic analyzer. The reciprocating can comprise electrically controlling the pump piston movement.

According to various embodiments, a method is provided that can comprise reciprocating a pump piston in a first direction to cause fresh fluid to be received into a chamber, and reciprocating the pump piston in a second direction to cause the fresh fluid to exit the chamber and enter a single capillary or multi-capillary array of a capillary electrophoretic analyzer. In some embodiments, the reciprocating can comprise controlling the pump piston movement using a programmable controller adapted to control a current to a stepper motor.

These and other features of the present teachings are set forth herein. It is to be understood that both the foregoing general description and the following detailed description are exemplary and explanatory only and are intended to provide a further explanation of the present teachings, as claimed.

### BRIEF DESCRIPTION OF THE DRAWINGS

The skilled artisan will understand that the drawings described below are for illustration purposes only. The drawings are not intended to limit the scope of the present teachings in any way.

FIG. 1 is a system block diagram of a multi-capillary electrophoresis system according to various embodiments;

FIGS. 2a through 2c illustrate a detector assembly according to various embodiments;

FIG. 3 is a detailed block diagram of a polymer delivery pump system according to various embodiments;

FIG. 4 is an assembly diagram showing the connections to an upper polymer block and a lower polymer block according to various embodiments;

FIG. 5 is a cross-sectional view of a polymer-delivery pump according to various embodiments;

FIG. 6 is a cross-sectional exploded view of a polymer-delivery pump according to various embodiments;

FIGS. 7a and 7b illustrate a water trap flush and fill procedure in accordance with various embodiments; and

FIGS. 8a and 8b are a flow chart of a fluid charging method according to various embodiments.

### DESCRIPTION OF VARIOUS EMBODIMENTS

The present teachings relate to methods and apparatus for manipulating separation media. In various embodiments, for example, the present teachings provide a polymer delivery pump and system and method useful, among other things, for filling one or more electrophoresis channels, or capillaries, with a separation medium such as, for example, a flowable sieving polymer.

A Polymer-Delivery Pump (PDP), according to various embodiments of the present teachings, provides users with an easier, more automated way to install polymer onto a capillary electrophoresis system as compared to prior syringe-based systems. A PDP can, in various embodiments, provide for a more streamlined workflow and may reduce the downtime from issues attributed to a syringe system.

The present teachings can be employed in connection with a variety of electrophoresis systems. As non-limiting examples, the present teachings can be adapted for use in connection with methods and apparatus such as those described in patent publications Nos. WO2003/027028 A1, US2003/0221965 A1, US2001/0040095 A1, US2003/0226756 A1, US2003/0127328 A1, US2004/0000481 A1, US2003/0201180 A1, US2004/0018638 A1, US2001/0040094 A1, US2002/0023839 A1, US2002/0003091 A1, and US2002/0179446 A1, each of which is hereby incorporated herein by reference.

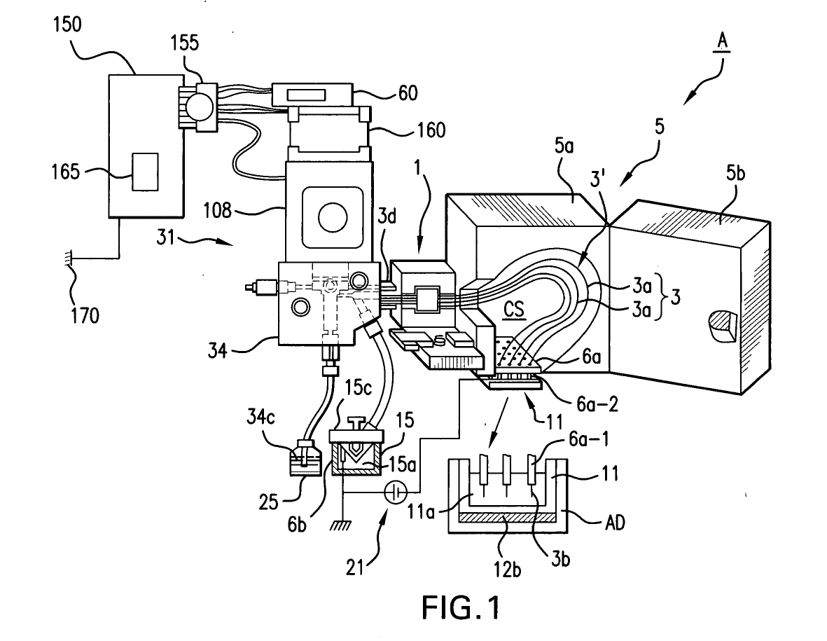

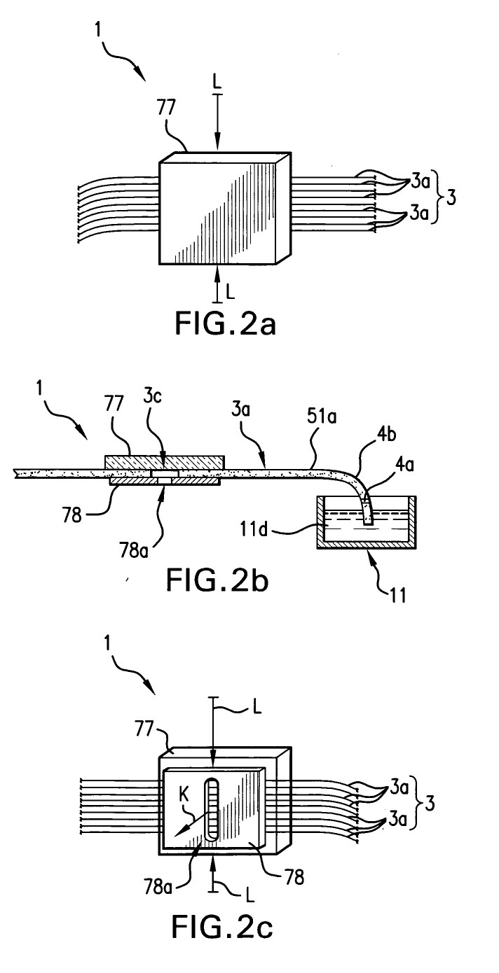

FIGS. 1-4 illustrate a multi-capillary electrophoresis system according to various embodiments. As shown, the multi-capillary electrophoresis system can comprise a single capillary or multi-capillary array 3 including plural capillaries 3a installed in a container portion CS of a temperature regulated chamber 5. The single capillary or multi-capillary array 3 can comprise plural, for example, 96 capillaries 3a. A sample 4a (FIG. 2b) containing, for example, specimens of DNA molecules, and an isolation medium 4b (FIG. 2b) functioning as a medium for isolating the DNA molecules in the sample 4a, can be filled in the capillaries 3a. In various embodiments, the isolation medium 4b can comprise, for example, a polymer in a gel form. The DNA fragment sample contained in the sample 4a can be distinguished by labeling the primer or the terminator with a fluorescent substance using the Sangar dideoxy method. The DNA fragment sample thus labeled with a fluorescent substance can be distinguished by the optical means as described, for example, in U.S. Published Patent Application No. US 2003/0102221 A1, filed Sep. 18, 2002 and assigned U.S. application Ser. No. 10/245,492 entitled, “MULTI-CAPILLARY ELECTROPHORESIS APPARATUS,” the disclosure of which is hereby incorporated by reference at least with respect to its teachings regarding an electrophoresis apparatus and temperature control thereof.

According to various embodiments, one end of the capillary 3a can comprise an injecting end 3b for injecting the sample 4a (as shown in FIG. 2b) by protruding the injecting end 3b from a bottom of the temperature regulated chamber 5. The injecting end 3b can be immersed in a buffer solution 11a. The buffer solution 11a can be contained in a buffer container 11. Electrodes 6a-1 can be mounted on or near the injecting ends 3b of the capillaries 3a. The electrodes 6a-1 can be made in electrical contact with an electrode plate 6a comprising, for example, an electrically conducting material. The electrode plate 6a can comprise, for example, copper, stainless steel, or an electrically conductive filled rubber material. In various embodiments, the electrode plate 6a on the side of the injecting ends 3b can be formed by pressing stainless-steel or platinum tubes 6a-1 into a metallic plate 6a-2, for example, into a metal material plate or another electrically conducting material plate. Other embodiments are also possible.

The injecting end 3b can be inserted in the stainless-steel tubes 6a-1 to integrate the sample injecting end 3b and the electrode plate 6a. A positive electrode of a direct current can be connected to the electrode plate 6a through an electrode (not shown in the figure) of the system. The isolation medium 4b can be filled in the capillaries 3a, and the sample 4a can be filled in the vicinity of the injecting end 3b. The injecting end 3b and the electrode 6a can be immersed in the buffer solution 11a filled in the buffer container 11. In various embodiments, the buffer solution 11a can comprise, for example, TBE (a mixed solution of tris(hydroxymethyl)aminomethane, boric acid and EDTA (ethylenediaminetetraacetic acid)) or TAPS (N-tris(hydroxymethyl)methyl-3-aminopropanesulfonic acid).

The buffer container 11 can be installed in an adapter AD. The adapter AD can comprise a rubber heater 12b, a thin film heater, a KELVAR sandwich heater, a Peltier heating device, or the like, disposed on the inner bottom surface thereof.

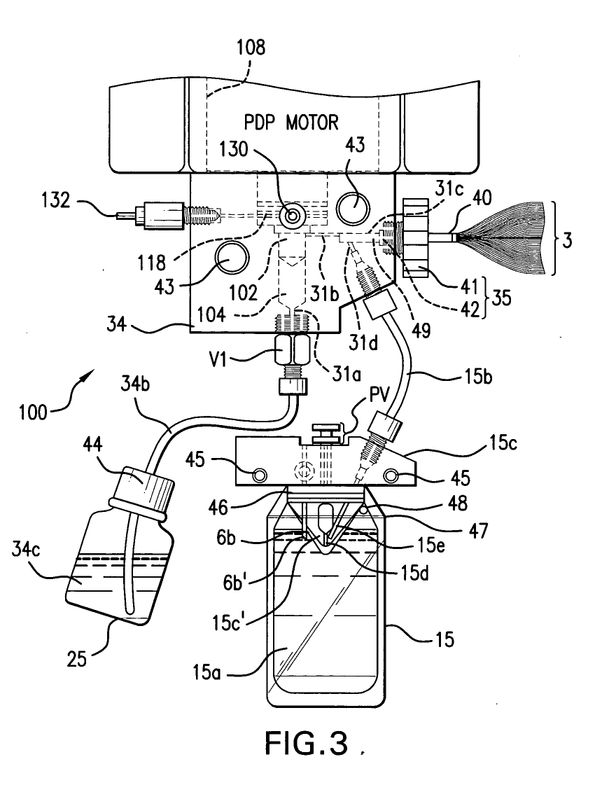

According to various embodiments, the other end of the capillary 3a can protrude from a side opening of the temperature regulated chamber 5 and through a detector assembly 1 for detecting components of the sample 4a. A plurality of these end parts 3d can be packed at a capillary fixing part or connector 35, which can comprise, for example a ferrule 42 and knob 41 assembly. End parts 3d can be connected to a block, for example, an upper polymer block (also referred to as a pump block) 34. For example, the capillary array end parts 3d, or tip 40 (FIG. 3), can comprise, according to some embodiments, a sealed, dense bundle of capillaries, which can be fitted by a user with the threaded array knob 41 and double-tapered ferrule 42, which together form a high-pressure seal when the array knob is attached to the polymer block 34. In some embodiments, the system can connect to a single capillary as opposed to a multi-capillary array.

In various embodiments, the upper polymer block 34 can be connected to a buffer storage container (for example, a buffer jar) 15 holding a buffer solution 15a therein, a polymer storage container (for example, a polymer bottle) 25 holding a polymer, for example, separation medium, 34c therein, and a Polymer-Delivery Pump (PDP) 31. The polymer storage container 25 can comprise a supply bottle and a bottle cap 44 with a hole permitting passage of a polymer supply tube therethrough.

In various embodiments, the capillary electrophoresis system A can separately control the temperature of different components or regions of the capillary electrophoresis system A. For example, a thermostat oven can be provided to contain one or more electrophoresis capillaries. A housing can be provided to contain at least one of the pump block 34, the buffer storage container 15 and the PDP 31. Further details regarding temperature control for the capillary electrophoresis system A are described in U.S. Published Patent Application No. US 2003/0102221 A1, the disclosure of which has been incorporated herein by reference. For example, in various embodiments the multi-capillary electrophoresis system A can comprise one or more temperature controlling parts (not shown) for controlling the temperature of the capillaries 3a with the thermostat oven (temperature-controlled chamber) 5 of the container portion CS.

In various embodiments, PDP 31 can comprise a pump displacement motor housing 108, a stepper motor 160, an encoder 60, and a controller 150. The stepper motor 160 can be coupled to the pump displacement motor housing 108. The stepper motor 160, pump displacement motor housing 108, and the encoder 60 can be coupled to the controller 150 using a controller connector 155. The stepper motor 160 can be coupled to the pump displacement motor housing 108 with a screw drive to convert rotational movement to translational movement. In various embodiments, the controller 150 can comprise electrical devices and components contained on a Printed Circuit Board (PCB). In various embodiments, the controller 150 can be provided in communication with a computing system (not shown) via a computer/network interface 170. In various embodiments, the computer/network interface 170 can be, for example, an Ethernet interface. In various embodiments, the controller 150 can be configured to control the operation of the stepper motor 160 and the pump displacement motor housing 108 of the PDP 31 for polymer fill operations as described herein. In various embodiments, the controller 150 can comprise firmware 165 in which is embodied a sequence of programmed instructions that, when executed by the controller 150, cause the controller 150 to perform the operations described herein. In some embodiments, the controller 150 can respond to commands received via the computer/network interface 170. The controller 150 can output status and other information via the computer/network interface 170. In various embodiments, the controller 150 can be coupled to an encoder 60 for monitoring operation of the stepper motor 160.

FIGS. 2a through 2c illustrate a detector assembly according to various embodiments. With regard to FIGS. 2a through 2c, there is shown a detector assembly 1. According to various embodiments, one or more samples (such as sample 4a) comprising, for example, one or more analytes such as DNA molecules, proteins and/or peptides, and a separation medium 4b, such as a flowable polymer for sieving the molecules in the sample 4a, can be introduced into the capillaries 3a. The separation medium 4b can comprise, for example, a polymer. For example, the polymer can comprise one or more of linear polyacrylamide, various derivatives of cellulose (e.g., MC, HPMC, etc.), galactomannan, glucomannan, polyvinyl alcohol, polyethyleneoxide, agarose, dextran, polydimethylacrylamide, polyhydroxypropylacrylamide, and/or polyacryloylethoxyethanol. In some embodiments, the polymer is a member of the POP™ polymer family, such as POP-4, POP-5, POP-6, or POP-7 available from Applied Biosystems of Foster City, Calif. In some embodiments, the polymer can comprise one of those disclosed in U.S. Pat. Nos. 5,427,729; 5,181,999; 5,015,350; 5,164,055; 5,126,021; 5,264,101; 5,759,369; 5,468,365; 5,290,418; 6,051,636; 5,891,313; 5,374,527; 5,916,426; 6,355,709 B1; 5,567,292; 6,358,385 B1; 6,297,009 B1; 5,578,179; and 6,706,162 B1, each of which is hereby expressly incorporated herein by reference, and/or in U.S. patent applications Ser. Nos. 10/843,114 and 10/629,524, each of which is hereby expressly incorporated herein by reference. In various embodiments, the polymer can have a viscosity of at least twice the viscosity of water. In some embodiments, the polymer can have a viscosity of from about 150 to about 550 times the viscosity of water, for example, from about 150 to about 300 times the viscosity of water.

According to various embodiments, DNA fragments contained in the sample 4a can be distinguished by labeling a primer or a terminator with a fluorescent substance such as, for example, a dye. Examples of such fluorescent dyes include, but are not limited to, the 5FAM, JOE, TAMRA, and/or ROX dyes available from Applied Biosystems of Foster City, Calif. Distinguishing of DNA fragments in this manner can be accomplished, for example, using the Sangar dideoxy method. The labeled DNA fragments can be detected utilizing a suitable optical system. According to some embodiments, a light source or light emission component L such as, for example, a laser or Light Emitting Diode (LED) emits radiation (e.g., light) that excites the fluorescent dyes of the DNA fragments. A Charge Coupled Device (CCD) or photodiode can be provided for receiving the light K then emitted by the fluorescent dyes.

As shown in FIGS. 2a through 2c, the single capillary or multi-capillary array 3 formed with plural capillaries 3a can be supported by clamping between a capillary supporting component 77 comprised of, for example, a glass or plastic plate, and a pressing member 78. An outer periphery of the capillaries 3a can be covered with a light shielding resin 51a, such as polyimide. A region 3c between the capillary supporting part 77 and the pressing member 78 can have the resin 51a removed to facilitate excitation and/or emission.

According to various embodiments, the region 3c can be irradiated with laser or other suitable light (e.g., LED light), L. This region 3c is referred to as a detecting component. An opening 78a can be formed in the region containing the detecting component 3c in the pressing member 78. Emission light K generated upon irradiating the sample with the laser light can be radiated to the exterior through the opening 78a. The structures described herein are collectively referred to as a detector assembly denoted by the reference numeral 1.

According to various embodiments, fluctuation in intensity depending on the position of the light incident on the capillaries 3a can be suppressed by irradiating the capillaries 3a with the light from opposed sides (e.g., both above and beneath) as shown in FIGS. 2a and 2c.

FIG. 3 is a detailed block diagram of a polymer delivery pump system 100 according to various embodiments. With regard to FIG. 3, the polymer delivery pump system 100 can comprise the upper polymer block 34, the polymer-delivery pump 31 connected to the upper polymer block 34, and a lower polymer block 15 connected to the upper polymer block 34. The polymer delivery pump 31, polymer storage container 25 and buffer storage container 15 can be connected to the upper polymer block 34. Flow paths, such as 31a to 31d, can be formed in the upper polymer block 34. When the valve PV is closed, the flow will instead travel through tip 40 into the single capillary or multi-capillary array 3. In some embodiments, a single block can be used instead of an upper block and a separate lower block.

The polymer-delivery pump 31 can comprise the pump displacement motor housing 108 and the pump piston 102. The upper polymer block 34 can comprise a fluid chamber or pump chamber 104 adapted to receive the pump piston 102 and to permit reciprocating movement of the pump piston 102 therein. A water trap 118 can be formed in the upper polymer block 34 by a first seal 120 and a second seal 122. In various embodiments, the seals 120 and 122 can be annular seals that surround the pump piston 102 in the upper polymer block 34.

In various embodiments, the upper polymer block 34 can comprise, for example, a block formed of an acrylic resin. The upper polymer block 34 can be adapted to receive the pump piston 102 into the chamber 104. The upper polymer block 34 can comprise flow paths 31a through 31d, an array port 49, and mounting pins 43. The upper polymer block 34 can also comprise a Luer® fitting 130 to provide access to the water trap 118, and an exit port for draining water from the water trap 118 via an exit port fitting 132.

A capillary array tip 40 of the single capillary or multi-capillary array 3 can be connected to the upper polymer block 34 at the array port 49 using the connector 35 which can comprise, for example, a double-tapered ferrule 42 and knob 41 assembly.

The lower polymer block 15c can be connected to the upper polymer block 34 via the flow path 15b. The flow path 15b can comprise interconnect tubing fixedly attached to the upper polymer block 34 and the lower polymer block 15c. In various embodiments, the flow path 15b can comprise a sidewall that exhibits a surface energy of about 30 dynes/centimeter or more. The lower polymer block 15c can comprise a pin valve PV and mounting pins 45. In various embodiments, the buffer storage container 15 holding a buffer solution 15a therein can be fixedly attached to the lower polymer block 15c. The buffer storage container 15 can comprise a buffer fill line 47 and an overflow hole 48. The lower polymer block 15c can comprise an O-ring seal 46 for forming a leak-free seal between the lower polymer block 15c and the buffer storage container 15. The lower polymer block 15c can also comprise a flow path 15e, a protrusion part 15c′ protruding downward with respect to the lower polymer block 15c, and a pin valve PV for opening and shutting an end opening 15d of the flow path 15e. A tip end of the pin valve PV can reach the interior of the protrusion part 15c′. The lower polymer block 15c can also comprise an electrode 6b further comprising a tip end 6b′.

The polymer storage container (for example, a polymer bottle) 25 holding a polymer, for example, separation medium, 34c therein, can be connected to the upper polymer block 34 using a flow path 34b. The flow path 34b can comprise interconnect tubing fixedly attached to the upper polymer block 34 and to the polymer storage container 25 through a bottle cap 44 with a hole permitting passage of a polymer supply tube therethrough. According to various embodiments, fresh polymer 34c can be held in the polymer storage container 25. The polymer storage container 25 can be connected to an end of a flow path 31a via the flow path 34b (also referred to as a polymer supply tube) using a polymer storage container connector. In various embodiments, the polymer storage container connector can comprise a first valve (check valve) V1 can be provided between an end of the flow path 34b and the flow path 31a to allow one-way flow of the polymer from the polymer storage container 25 to the upper polymer block 34.

According to various embodiments, when a pin valve PV (also referred to as a buffer valve) at a lower polymer block 15c (described below) is closed, and a piston 102 of the polymer-delivery pump 31 is withdrawn or reciprocated in a first direction to expand the volume of a chamber 104, thereby reducing pressure, fresh polymer 34c in the polymer storage container 25 can be filled or drawn into the chamber 104 (also referred to as an internal bore) of the polymer-delivery pump 31 via the tube path 34b and the flow path 31a. When the piston 102 is aspirating, valve PV can be closed and the array can be maintained in water, in a buffer solution, or in another electrically conducting liquid. When the pin valve PV is closed, and the piston 102 of the polymer-delivery pump 31 is moved or reciprocated in a second direction to reduce the volume of the chamber 104, the fresh polymer in the chamber 104 can be forced out of an opening of the chamber and injected into the capillaries 3a through the flow path 31b and a flow path 31c.

The controller 150 can comprise a computing device such as, for example, a processor, microprocessor, or microcontroller device that executes a sequence of programmed instructions. The controller 150 can further comprise a sequence of programmed instructions that, when executed by the processor, microprocessor, or microcontroller device, cause the device to be configured to perform the operations described herein. In various embodiments, the sequence of programmed instructions can be stored or embodied in the firmware device 165. In various embodiments, the firmware 165 can be, for example, a Programmable Logic Array (PLA). Other embodiments are possible. For example, the instructions can be stored or encoded using a Read Only Memory (ROM), Programmable ROM (PROM), Erasable PROM (EPROM), or similar such device that provides non-volatile storage. The instructions can be read into non-volatile memory such as, for example, Random Access Memory (RAM), at time of execution, although in other embodiments, the instructions are not read into such a memory. In various embodiments, the instructions can be implemented using a programming language such as, for example, the Standard Commands for Programmable Instrumentation (SCPI) standard programming language. SCPI comprises a standard set of commands to control test and measurement devices. In various embodiments, the sequence of programmable instructions can cause the controller 150 to reciprocate the pump piston 102 and open and close valves as described above to perform the polymer fluid charging operations described herein.

In various embodiments, the controller 150 can maintain a constant fluid pressure level in the polymer flow path 31a through 31d during the drawing and filling operations described above by controlling the speed of movement of the pump piston 102 by controlling the drive current provided to the stepper motor 160. In various embodiments, the PDP 31 output pressure can be monitored at production time (such as, for example, at the factory) to determine the stepper motor 160 current value that generates 1000 psi. In various embodiments, the stepper motor 160 current value that generates 1000 psi can vary among different units from about 0.18 Amps to 0.35 Amps. This current value can be stored in a file and provided to the controller 150 at startup initialization. In various embodiments, the current value can be stored in the “calib.ini” file (for example, calibration initialization file). In operation, the controller 150 can cause the amount of current equal to the current value received from the calib.ini file to be provided to the stepper motor 160. Upon achieving a pressure in the polymer flow path of about 1000 psi, the rotational movement of the stepper motor 160 can be stopped. As polymer is pushed into the capillaries, the pressure in the polymer flow path will be reduced and the stepper motor 160 can start again until the pressure reaches 1000 psi, at which point the stepper motor 160 stalls. This process can be completed until the capillary fill operation is complete. In various embodiments, the capillary array can be filled with polymer 1.5 times the array volume to ensure that prior traces of DNA in the old polymer are flushed from the system, as well as to accommodate slight flexion in the interconnect tubing.

In various embodiments, the controller 150 can monitor the speed of the pump piston 102 to detect a leak condition. For example, if the controller 150 determines that the pump piston movement exceeds a predetermined threshold, the controller 150 can stop the pump piston 102 and report an error or leak condition to alert an operator to take corrective action.

In various embodiments, the controller 150 can be provided in communication with a computing system (not shown) via the computer/network interface 170. In various embodiments, the computer/network interface 170 can comprise an Ethernet interface. The controller 150 can communicate with a standalone computer or with one or more networked computers. In various embodiments, the controller 150 can accept human operator input via the interface 170 from a keyboard, mouse, or other such input and selection device. The controller 150 can also output status information to the human operator using a display of a computer via the interface 170. In various embodiments, the display can be a Graphical User Interface (GUI). In various embodiments, the controller 150 can exchange information over the interface 170 in accordance with the Transport Control Protocol/Internet Protocol (TCP/IP). The controller 150 can, in various embodiments, exchange information in the form of interactive pages such as, for example, HyperText Markup Language (HTML) formatted pages using the HyperText Transport Protocol (HTTP).

BioMonitor software service (Applied Biosystems, Foster City, Calif.) can be used to remotely monitor the system over the internet, for example, by technical support or field service personnel.

In various embodiments, the polymer can comprise the separation medium 4b inside the capillaries 3a. The separation medium 4b, after one or more electrophoretic runs, can be discharged from the capillaries 3a through the injecting end 3b of the capillaries by again charging the capillaries with fresh polymer in accordance with the foregoing operation. According to various embodiments, the separation medium 4b and the sample 4a can be discharged out through the injecting end 3b. According to various embodiments, the separation medium 4b can be replaced per analysis of one sample, and a fresh separation medium 4b can be used for analysis of another sample.

According to various embodiments, a tube path 15b (also referred to as an interconnect tube) can be provided between a flow path 31d in the upper polymer block 34 and a flow path 15e of the lower polymer block 15c to connect them for fluid communication. The lower polymer block 15c can comprise a protrusion part 15c′ protruding downward. The pin valve PV, for opening and shutting an end opening 15d of the flow path 15e, can be supported in the lower polymer block 15c. A tip end of the pin valve PV can reach the interior of the protrusion part 15c′. The separation medium 4b can be filled in the flow path 31d in the upper polymer block 34, the tube path 15b, and the flow path 15e in the lower polymer block 15c. Buffer solution 15a can be filled in the buffer storage container 15. Separation medium 4b can be placed in the buffer storage container 15 as an alternative or in addition to the buffer solution. The separation medium 4b and the buffer solution 15a can be in contact with each other at the end opening 15d of the flow path 15e.

During electrophoresis, the pin valve PV can be moved to the withdrawing direction (upward in the figure) to provide a conductive pathway through valve PV. A tip end 6b′ of an electrode 6b can be grounded. Upon opening the pin valve PV, an electrification path can be formed between the electrode 6a and the electrode 6b through the (a) buffer solution 11a (between the electrode 6a and the sample injecting end 3b of the capillaries), (b) the separation medium 4b (filled in the sample injecting end 3b of the capillaries), (c) the capillaries 3 through the end part 3d thereof, (d) the flow path 31d in the upper polymer block 34, (e) the tube path 15b and the flow path 15e in the lower polymer block 15c, and (f) the buffer solution 15a (between the end opening 15d of the flow path 15e and the electrode 6b).

Therefore, when the pin valve PV is opened, and a voltage is applied between the electrode 6a and the electrode 6b with a direct current power supply 21 (reference FIG. 1), such a voltage can be applied between both the ends of the electrification path; that is, a voltage can be applied to the buffer solutions positioned on both ends of the separation medium, which are present along the electrification path. Consequently, an electric current can be created in the separation medium 4b in the capillaries 3a.

The pin valve PV can be closed when the polymer is replaced in the capillaries 3a. At this time, the separation medium can be injected from the polymer storage container 25 to the capillaries 3a using the polymer delivery pump 31.

In various embodiments, the pin valve PV can comprise a solenoid (not shown) for opening and closing the pin valve PV in response to an electrical signal from the controller 150.

Any suitable electrophoresis buffer can be employed (e.g., TAE, TBE, TPE, etc.). According to various embodiments, the buffer solution 11a and the buffer solution 15a can be prepared with, for example, a sodium ion and TAPS (N-tris(hydroxymethyl)methyl-3-aminopropanesulfonic acid). The tube path can be similarly immersed in the buffer solution 15a.

According to various embodiments, the buffer solutions 11a and 15a can be placed in the buffer containers 11 and 15, respectively. The electrode 6a and the electrode 6b can be immersed in the buffer solution 11a and the buffer solution 15a, respectively. The buffer solutions 11a and 15a can connect electrically between electrodes and the separation medium in the capillaries.

An upper surface of the buffer solution 15a can be positioned above an end opening 15d of the flow path 15e. Therefore, at least a part of the protrusion part 15c′ of the lower polymer block 15c can be immersed in the buffer solution 15a.

In various embodiments, one or both of the polymer supply tube 34b and the interconnect tube 15b can comprise a tube having an inner diameter (ID) greater than 60 thousandths of an inch. In some embodiments, the ID can be from about 0.5 mm to about 3 mm (for example, between about 1-2 mm; and in certain embodiments about 1.57 mm). The tubing can comprise a material that offers high burst pressure and good chemical compatibility. In various embodiments, the tubing can comprise RADEL® tubing. In some embodiments, the tubing is transparent or semi-transparent, so that users can ascertain visually whether or not bubbles are present therein. The present teachings provide a system that avoids spatially restricted areas (for example, using large-ID tubing), which can help to minimize localized polymer hot spots as could otherwise occur if bubbles are formed. In various embodiments, the channels, flow paths, chambers, etc. within the upper and lower blocks have IDs at least as great as the ID of the interconnect tubing.

According to various embodiments, the separation medium 4b can be filled in the capillaries 3a using the polymer-delivery pump 31. For example, 1, 2, 4, 8, 16, 32, 48, 96, 384, or more capillaries (3a) can be used. Subsequently, a sample 4a containing one or more analyte molecules (e.g., a polynucleotide sample, such as a sample comprising DNA fragments of varying lengths) can be introduced to the capillaries 3a through their injecting ends 3b. The injecting ends 3b can be immersed in the buffer solution 11a held in the buffer container 11.

A voltage, for example, about from 7.5 kV to 20 kV (e.g., 10 kV), or more, can be applied between the electrode 6a (cathode) and the electrode 6b (anode) with the direct current power supply 21 (reference FIG. 1). Consequently, the DNA molecules will migrate toward the electrode 6 (electrophoresed) due to their negative charge. Differences in the electrophoretic migration velocity of the DNA molecules occur corresponding to the base lengths thereof. The molecules having smaller base lengths exhibit larger electrophoresis migration velocities thereby requiring shorter periods of time to reach the detecting part 3c. Upon irradiating the sample (e.g., DNA molecules) reaching the detecting part 3c with light L, identification markers (e.g., fluorophores) attached to the analyte molecules can be excited to cause detectable emission (e.g., fluorescence). The emission can be collected and imaged onto a sensing device, such as a CCD image sensor provided in a CCD camera. According to various embodiments, DNA molecules can be distinguished by electric signals obtained from the CCD camera, and thus the DNA can be analyzed. Consequently, a sample containing DNA fragments can be subjected to electrophoresis, and fluorescence from the sample can be detected in the course of electrophoresis, whereby DNA base sequencing can be carried out for determining the base sequence.

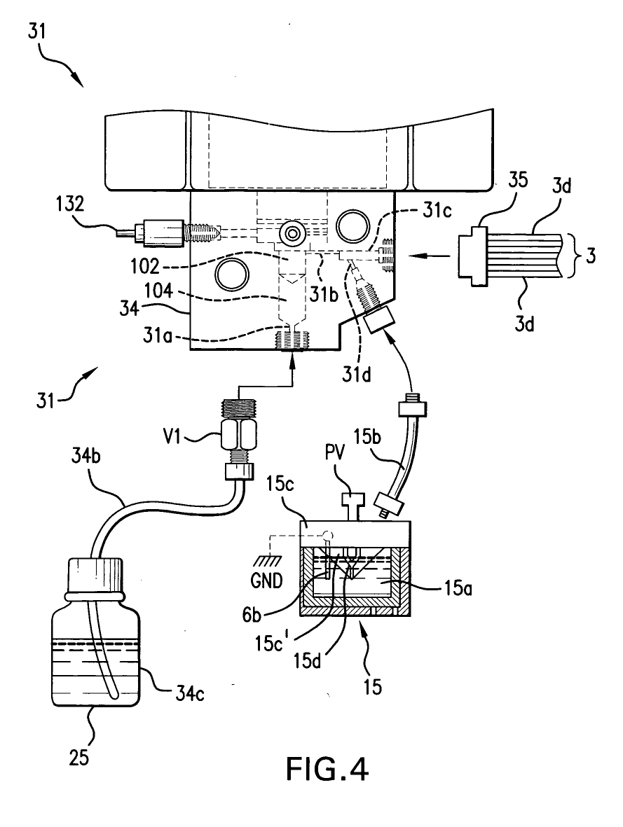

FIG. 4 is an assembly diagram showing the connections to the upper polymer block 34 and the lower polymer block 15c according to various embodiments. With regard to FIG. 4, there is shown the polymer storage container 25 to be connected to an end of a flow path 31a via the flow path 34b and check valve V1, a tube path or flow path 15b (also referred to as an interconnect tube) to be connected between a flow path 31d in the upper polymer block 34 and a flow path 15e of the lower polymer block 15c for fluid communication, and the single capillary or multi-capillary array 3 to be connected to the upper polymer block 34 using the connector 35.

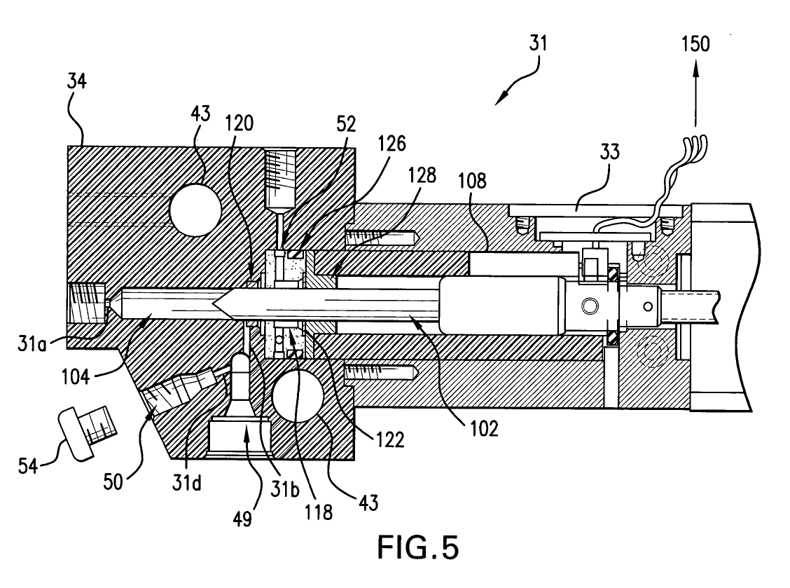

In various embodiments, the polymer-delivery pump 31 can comprise a positive displacement reciprocating piston pump assembly. FIG. 5 is a cross-sectional view of the polymer-delivery pump 31 according to various embodiments. With regard to FIG. 5, there is shown the pump piston 102, the chamber 104, the pump displacement motor housing 108, the upper polymer block 34, and the connector to the controller 150. In various embodiments, the polymer-delivery pump 31 can comprise the pump displacement motor housing 108 and the pump piston 102. The upper polymer block 34 can comprise a pump chamber 104 adapted to receive the pump piston 102 and to permit reciprocating movement of the pump piston 102 therein. The water trap 118 can be formed in the upper polymer block 34 by the first seal 120 and the second seal 122. In various embodiments, the seals 120 and 122 can be annular seals that surround the pump piston 102 in the upper polymer block 34. The upper polymer block 34 can also be adapted to receive the check valve V1, the single capillary or multi-capillary array 3 via the array port 49, the interconnect tube of the flow path 15b via an interconnect tube port 50, and the exit port 132 via the water port 52. Plugs 54 can be inserted into these openings of the upper polymer block 34 to prevent contamination during shipping.

The pump displacement motor housing 108 can be coupled to the stepper motor 160 and connected to the pump piston 102 and configured to cause reciprocating movement of the pump piston 102 in response to electrical signals to the stepper motor 160. In various embodiments, the electrical control signals can be received from the programmable controller 150. The pump displacement motor housing 108, stepper motor 160, pump piston 102, and encoder 60 can be obtained form various manufacturers such as, for example, the Confluent™ products available for Scivex Corporation of Oak Harbor, Wash.

In various embodiments, the polymer-delivery pump 31 can comprise a piston 102 adapted for reciprocal movement within a pump chamber 104. The polymer-delivery pump 31 can have, for example, a capacity of several milliliters (e.g., 2, 3, 4 or 5 milliliters), or 1 milliliter, or less. In some embodiments, for example, the polymer-delivery pump has a capacity within a range of from about 250 to about 750 μL (e.g., a 300, 400, 500, 600 or 700 μL capacity). The polymer-delivery pump 31 can comprise the pump displacement motor housing 108, stepper motor 160, and encoder 60, to move the piston 102 within the upper block 34. In various embodiments, the encoder 60 may be an optical encoder. In some embodiments, the polymer block 34 comprises an acrylic material. A small gap (e.g., about 15 thousands of an inch) can separate the outer surface of the piston 102 and the wall of the pump chamber 104.

In some embodiments, at the top of the polymer block 34, a high-pressure seal with a water trap 118 (also referred to as a water chamber) minimizes leakage of polymer into the motor. The water trap 118 can be defined, for example, by first and second seals, denoted in the figures as 120 and 122, which are spaced apart from one another. In various embodiments, the first seal (also referred to as the “bottom” seal) can comprise a “cup” seal disposed adjacent to the chamber 104, and the second seal (also referred to as the “top” seal) can comprise an Ultra-High Molecular Weight Polyethylene (UHMWPE), or a Viton® “cup” seal available from the DuPont Company of Wilmington, Del., PEEK™, and/or Buna-N. The water chamber 118 can further be defined by an intermediate piece 126, having an O-ring (e.g., a Buna-N O-ring) disposed thereabout. The intermediate piece 126 can be bounded above and below by the top and bottom seals, respectively. A top seal backup plate 128 can constrain the top seal from above.

In various embodiments, the PDP 31 can comprise a cam follower bearing (not shown) to prevent wear in a guide tube of the upper polymer block 34 caused by an anti-rotation pin of the pump displacement motor housing 108. In some embodiments, the anti-rotation pin can be replaced with a ball bearing and a pre-loaded spring to urge the bearing to contact one side of the guide tube as the bearing traverses the guide tube or slot.

As shown in FIG. 5, an LED limit sensor cover 33 can be provided to cover an LED limit sensor.

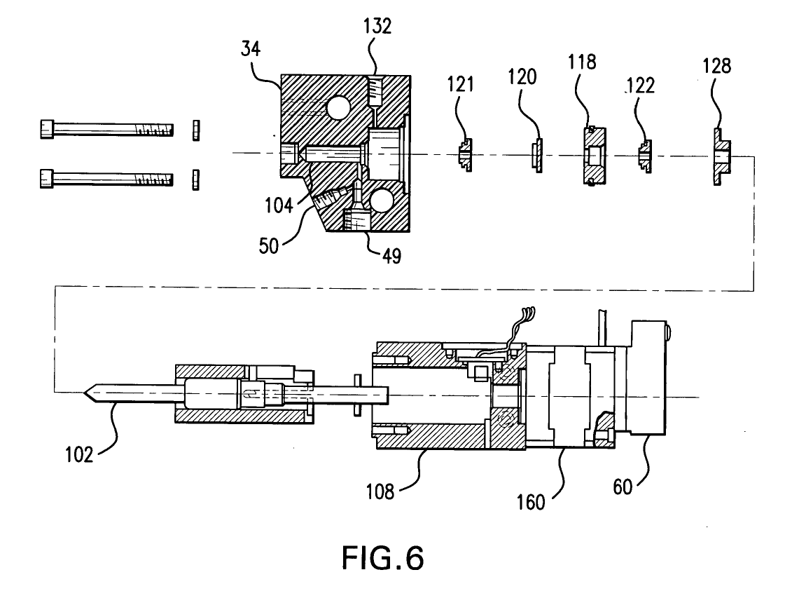

FIG. 6 is a cross-sectional exploded view of the polymer-delivery pump 31 according to various embodiments. With regard to FIG. 6, there is shown an order of assembly for the pump piston 102, the pump displacement motor housing 108, the stepper motor 160, the encoder 60, the first cup seal 121, the second cup seal 122, the water trap 118 (FIG. 6), and the top seal backup plate 128. As shown in FIG. 6, the upper polymer block 34 can be adapted to receive the pump piston 102, the first cup seal 121, the second cup seal 122, the intermediate piece 126, and the top seal backup plate 128 in the order indicated in FIG. 6, in various embodiments. A retainer 120 can be used to retain the cup seal 121 in an operable position. As shown in FIG. 6, the stepper motor 160 can be coupled to the encoder 60 for monitoring motor speed and providing motor status to the controller 150.

In some embodiments, both cup seals 121 and 122 can be comprised of UHMWPE. Viton® O-rings about the respective cup seals can provide an initial compression around the pump piston 102 shaft. While the cup seals 121 and 122 can generally be expected to function very well under pressure, before being pressurized, the O-rings can provide additional sealing.

In various embodiments, the Luer® fitting 130 can provide access to the water trap 118 from outside the pump 31, and an exit port can allow water to be drained from the water trap 118 via the exit port fitting 132. The water trap 118 can reduce or eliminate polymer leakage into the pump displacement motor housing 108. Periodically, the water trap 118 can be cleaned, as desired, by flushing water through the water seal Luer® 130 and exit fittings 132 on the upper polymer block. In some embodiments, in operation, the water trap 118 is entirely filled with Deionized (DI) water.

In some embodiments, as previously described, the pump block 34 can be connected to a lower polymer block 15c by an interconnect tube 15b. The lower polymer block 15 can incorporate an anode (electrode 6b), a buffer jar 15 (the buffer reservoir for the anode) and a buffer valve PV. With the buffer valve PV closed, the pump chamber 104 can be filled from the polymer bottle 25, through the polymer supply tube 34b (automatically opening the check valve V1) when the piston is retracted. The array 3 can be filled (after electronically closing the buffer valve PV) when the pump piston 102 is advanced closing the check valve V1. The buffer valve PV can be opened to flush the system with polymer or water, as desired.

In various embodiments, polymer (or water for cleaning) can be drawn into the chamber 104 from a supply bottle 25 through the polymer supply tube 34b and the check valve V1, which allows fluid flow only in the direction from supply 25 to chamber 104. In some embodiments, the buffer valve PV can be closed to fill the chamber 104; otherwise fluid can be drawn from the direction of the lower polymer block 15c. Since, in various embodiments, the capillary array 3 is generally open to outside pressure, fluid could also be drawn from the capillaries 3a. Since, in various embodiments, the narrow capillaries 3a can offer high resistance to fluid movement (for example, high pressure can be necessary for fluid motion in the capillaries) and since filling of the pump chamber 104 can occur at a pressure of less than one atmosphere (for example, 15 psi, 10 psi, or less), very little flow occurs in the capillaries 3a. In the event a new array 3 (empty) is used, the capillary tips (cathode ends) 3b can be placed in water, a buffer solution, or another conducting liquid, to ensure proper pump chamber 104 filling.

In various embodiments, to fill the capillaries 3a, the buffer valve PV can be closed and the pump piston 102 driven downward. The polymer flows past the descending piston 102 in the narrow (e.g., 15 thousands of an inch) gap region between the piston 102 surface and the chamber 104 inner walls) to the array port 49 and into the capillaries 3a. The capillaries 3a offer high resistance to fluid flow, but the polymer-delivery pump 31 can be configured to generate sufficient force (e.g., about 10 lbs, or greater) and pressure (e.g., about 20 psi or greater, about 100 psi or greater, or about 1000 psi, or greater) to fill the capillaries 3a with polymer. Controlling the current to the stepper motor 160 can control the piston 102 force. As discussed previously, the current for the stepper motor 160 of each pump 31 can be established individually at a separate test station during its manufacture. The current value for the pump 31 can be stored in an instrument .ini file (such as, for example, the calib.ini file) and automatically loaded when the instrument is booted up (along with other pertinent motor settings). In various embodiments, several commands can be used to move the piston 102 up and down. The command used to fill the array 3 can have the calibrated value to generate the 1000 psi. The encoder 60 can be monitored during the fill command by the controller 150. If the piston 102 moves faster than a preset value (stored in firmware, for example), the controller 150 can detect a leak or air bubble condition. If a leak or air bubble condition is detected, the controller 150 can stop the pump displacement motor housing 108 and output a “Leak Detect” error message to the operator.

In various embodiments, a coating (for example, Polyvinylpyrrolidone (PVP)) can be bonded to the surfaces (internal channels, flow paths, chambers, etc.) of the acrylic pump block 34 and the lower acrylic polymer block 15c to render the surfaces hydrophilic. The coating can be, for example, a covalently bonded PVP coating of, for example, about 1-2 μm thick.

The hydrophilic surfaces can serve, among other things, to prevent bubbles from sticking to the coated surfaces. Bubbles can cause problems such as: “Leak Detect” error, “Fluctuating Current” error, and/or arcing. Any one of these events can cause data loss and part failure. Air bubbles can compress during the array fill step when the buffer valve PV opens because the expanding air will pump the polymer into the buffer jar 15.

In some embodiments, the piston 102 surface can be plasma treated to render it hydrophilic. A hydrophilic surface can reduce the chances for bubbles to stick to the piston 102. Once the polymer wets the surface, it tends to stay wet. It can be advantageous to get polymer into contact with the piston 102 surface initially; if so, a hydrophilic surface can help to achieve this.

In various embodiments, the end of the piston 102 can be tapered (for example, into a near point) to reduce the possibility for bubbles from the polymer supply tube 34b to stick to the piston 102. Some embodiments can comprise a pointed end region for the piston 102 (for example, a cone shape). For example, the end region can terminate at a sharp point, or there can be a radius at the end of the point.

While the pump 31 can be disposed in any number of spatial orientations, various embodiments employ an orientation adapted to keep the piston vertical (or near vertical). The vertical orientation can help to avoid bubble formation/sticking.

In various embodiments, the pump piston 102 can comprise a material capable of achieving a smooth surface finish. For example, the piston 102 can have a surface comprising sapphire, ceramic, alumina, or another hard material, for example, having a hardness greater than that of alumina. The surface finish, according to various embodiments can be no greater than 3 μm, for example, about 1-2 μm Root Mean Square (RMS) surface finish. In various embodiments, the pump piston 102 can comprise a gemstone, for example, a sapphire or another gemstone, in order to achieve a 1-2 μm finish, which can minimize the wear to the cup seal. By this construction, nucleation sites for bubble formation can be minimized. With a smooth surface finish, as the piston 102 shaft travels past the seals, serration sites can be minimized. The sapphire or another gemstone can be synthetic and can be see-through, clear, and/or transparent such that air bubbles can be seen through it. The sapphire or another gemstone can be natural.

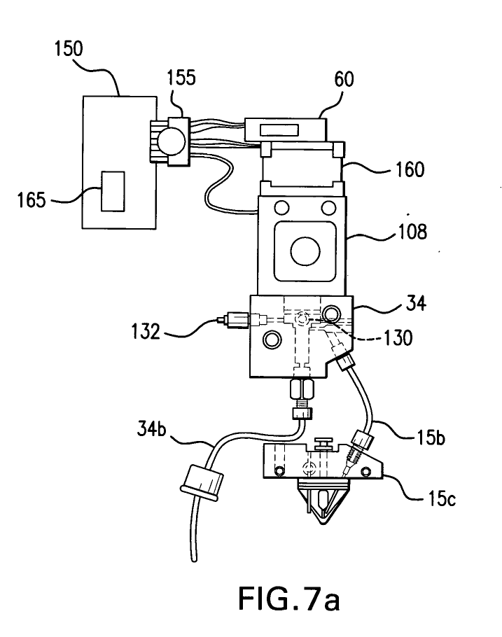

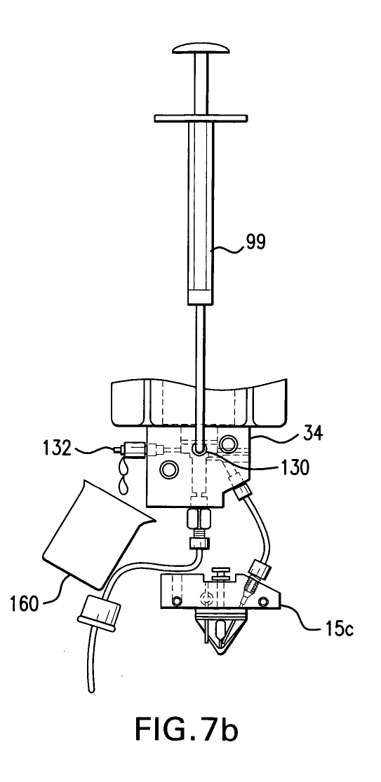

In various embodiments, the water trap 118 can be filled, or the water in the water trap 118 changed, in accordance with FIGS. 7a and 7b as follows. With regard to FIG. 7a, according to various embodiments, the water trap 118 can be provided behind the main chamber cup seal 120. The water trap 118 can reduce or eliminate the chance that some polymer gets by the cup seal 122. Polymer passing through the cup seal can, in some configurations, dry out and crystallize on the piston 102. For example, if the polymer has urea content, it could crystallize quickly. Crystallized polymer can cut the seal 120 or 122 about the piston 102 when the piston 102 bearing such crystallized polymer is reciprocated within the chamber 104. The water trap 118 can have cup seals at both ends (for example, seals 120 and 122).

With regard to FIG. 7b, the water chamber 104 can be manually filled and cleaned periodically (for example, once per month) by attaching a Luer® syringe 99 to the Luer® fitting 130, partially loosening the exit fitting 132 (for example, one-half turn) and the Luer® fitting 130 (for example, one-half turn), and then flushing DI water through the water trap 118. The flushing can flush out any water polymer that may have been trapped. A beaker 160 can be placed under the exit fitting 132 to catch water as it comes through the exit fitting 132. In various embodiments, the 5 to 10 mL of DI water can be sufficient to flush and fill the water trap 118. In various embodiments, a 20 mL beaker can be used to catch the water exiting the water trap 118. At the completion of the water trap 118 flush and fill, the Luer® fitting 130 and the exit fitting can be tightened and the Luer® syringe removed.

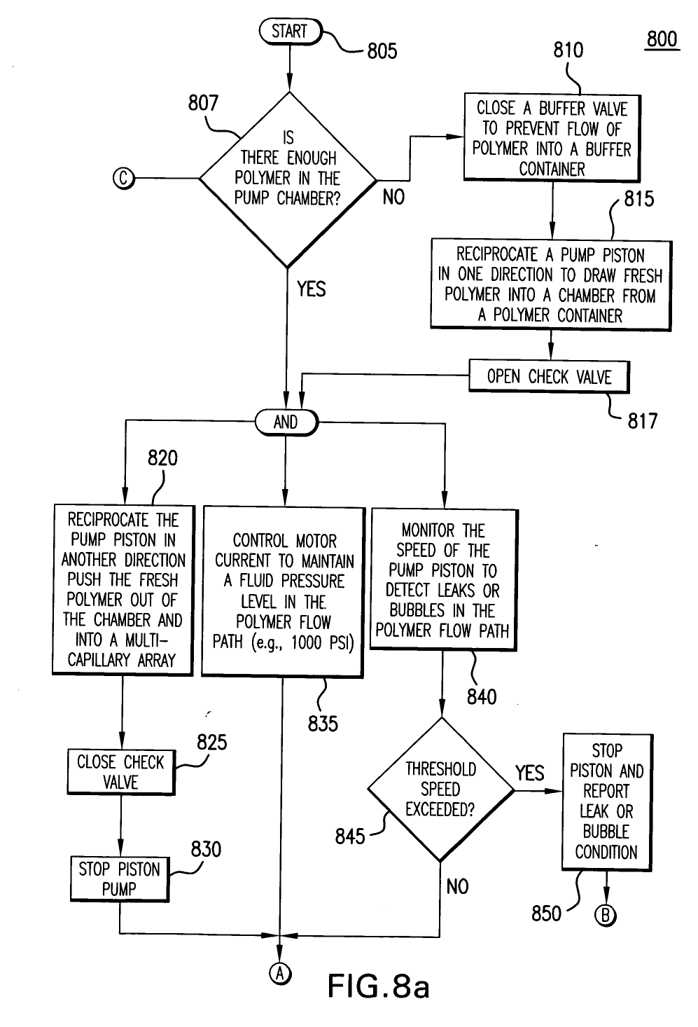

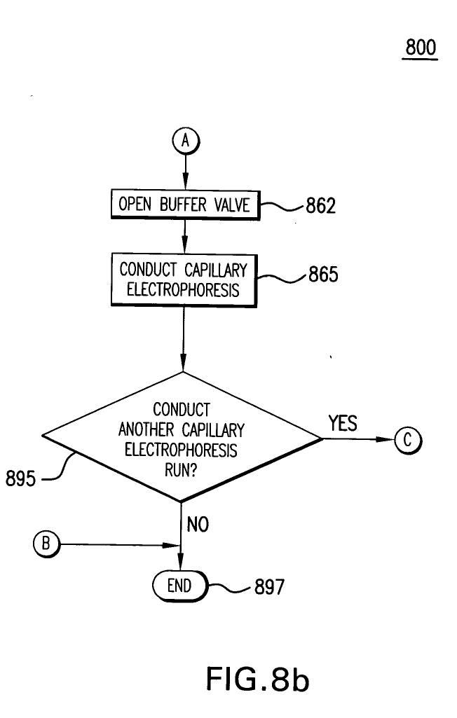

FIGS. 8a and 8b are a flow chart of a fluid charging method 800 according to various embodiments. As shown in FIG. 8a, fluid charging method 800 can commence at 805. At 807, if there is enough polymer in the pump chamber, the fluid charging method can continue. Alternatively, at 807, if there is not enough polymer, control can proceed to 810, at which the method can comprise closing a buffer valve to prevent flow of polymer into a buffer container. Control can then proceed to 815, at which the method can comprise reciprocating a pump piston in one direction to draw fresh polymer into a chamber from a polymer container. Control can then proceed to 817, at which the method can comprise opening a check valve. Control can then proceed to 820, 835, and 840. At 820, the method can comprise reciprocating the pump piston in another direction to push the fresh polymer out of the chamber and into a single capillary or multi-capillary array. Control can then proceed to 825, at which the method can comprise closing the check valve. Control can then proceed to 830, where the piston pump can be stopped. Processing can then continue to 862.

At 835, the method can comprise controlling a motor current to maintain a fluid pressure level in the polymer flow path (for example, 1000 psi). Processing can then continue to 862.

At 840, the method can comprise monitoring the speed of the pump piston to detect leaks or bubbles in the polymer flow path. From 840, control can proceed to 845 to determine if a threshold speed of the pump piston has been exceeded. If so, control can then proceed to 850, at which the pump piston can be stopped and a leak condition can be reported. If at 845, the method determines that the threshold speed has not been exceeded, then processing can continue to 862.

Following 830 and 835, where and if the method determines that the threshold speed for the pump piston has not exceeded a threshold at 845, control can then proceed to 862. With regard to FIG. 8b, at 862, the method can comprise opening the buffer valve. Control can then proceed to 865, where the method can comprise conducting capillary electrophoresis.

Control can then proceed to 897, the method can comprise determining whether or not to conduct another capillary electrophoresis run. If another capillary electrophoresis run is desired, then control can proceed to 897 as shown in FIG. 8a. If another capillary electrophoresis run is not desired, then control can proceed to 897 at which the method can end.

In some embodiments, a procedure to perform maintenance on the system can be provided. The maintenance can be performed manually and/or under a wizard control. Maintenance can comprise determining whether or not to change the fluid pressure level maintained by the pump piston when filling the single capillary or multi-capillary array. If a user desires to change the pressure level, the pump current value, specified in an instrument calibration file to effect a change in the fluid pressure level maintained by the pump piston, can be changed. If no change is desired, or after the change has been accomplished, the method can comprise determining the number of array fills that has been accomplished since the last chamber fill. If a desired number of array fills have been accomplished since the last chamber fill, the method can comprise washing the polymer path upon user command. If the user desires to wash the polymer path, the method can comprise conducting a polymer path wash using deionized water.

In an exemplary embodiment, a capillary electrophoresis system includes a polymer delivery pump having a chamber capacity of about 500 microliters (μL). The polymer delivery pump is designed to reliably deliver at least 50,000 injections (capillary array fills) without failure. The reliable PDP design reduces operating costs because consumables are reduced. Furthermore, the reliable PDP design provides a target Mean Time Between Failure (MTBF) that is useful for maintenance and capital expenditure planning. For example, the PDP designed to reliably delivery at least 50,000 injections has an MTBF of approximately five years if the capillary electrophoresis system is used 24 hours per day, 7 days per week, and 52 weeks per year to perform capillary electrophoresis runs.

In an exemplary embodiment, the PDP fills a 96×36 cm single capillary or multi-capillary array (single capillary or multi-capillary array having 96 capillaries of 36 centimeters capacity) in about 25 seconds. In this example, the single capillary or multi-capillary array utilizes about 234 μL of polymer to fill the single capillary or multi-capillary array with fresh polymer. The PDP can draw fresh fluid into and fill its 500 μL chamber, in about 40 seconds or less. Thus, filling of the PDP with fresh polymer normally occurs once for every two array fills. Depending on pump piston speed, filling the PDP can be performed faster or slower than 24 seconds. For example, the time to fill the PDP can be as little as 5 seconds or long as 90 seconds or more. The time to fill the PDP and the need to fill the PDP only once per every two array fills can reduce the operating time of the example system by about two minutes per capillary electrophoresis run compared to prior systems.

In an exemplary embodiment, a bubble removal cycle can utilize about 40 seconds to perform by an operator or service technician using the bubble purge wizard. Bubbles or their absence are readily observed by visual inspection of the interconnect tubing between the pump block and the lower polymer block. A bubble removal wizard can be executed using a computing device. The bubble removal wizard can comprise a sequence of programmed instructions that when executed by a processor cause the processor to control the piston pump to fill the capillary array with polymer in a manner that reduces the likelihood of air bubbles occurring in the polymer path. The bubble removal wizard can comprise outputting, using a display of the computing device, instructions to an operator or service technician for performing one more bubble removal cycles. The computing device can be provided in communication with the processor 150 using the connector 155, for example. The bubble removal wizard can comprise instructions for a discretionary array port flush and optional array fill. An operator or service technician can perform array installation using the array installation wizard in less than two minutes. An operator or service technician can perform array removal using the array removal wizard in less than two minutes. The wizard can provide predictable service times compared to prior systems in which service time is entirely technician dependent.

In an exemplary embodiment, washing of the polymer path can utilize about 5 minutes to perform by an operator or service technician using a polymer path wash wizard. In this example, DI water is used to wash the polymer path. Washing the polymer path can be accomplished at each polymer lot change or on a periodic basis such as, for example, once per month. The polymer path wash wizard can be executed using a computing device. The polymer path wash wizard can comprise a sequence of programmed instructions that when executed by a processor cause the processor to control the piston pump to wash the polymer path of the system with a cleansing fluid. The polymer path wash wizard can comprise outputting, using a display of the computing device, instructions to an operator or service technician for performing the polymer path wash. In an example, the polymer path wash method comprises: (1) replacing the polymer container 25 with a cleansing fluid container; (2) running the polymer path wash wizard; (3) replacing the cleansing fluid container with the polymer container; (4) continuing the polymer path wash wizard to replace the polymer. In an example, the cleansing fluid container is a bottle filled with DI water. The automated polymer path wash can reduce likelihood of contamination of the polymer path that could otherwise be caused by manual disassembly and washing of path components.

In an exemplary embodiment, the DI water in the water trap is replaced periodically such as, for example, once per month, manually using a syringe as described with respect to FIGS. 7a and 7b.

In an exemplary embodiment, the interconnect tubing used for the polymer flow path (such as, for example, the polymer flow path 15b, 15d, 15e, and 34b) has an Inner Diameter (ID) that is the same throughout the flow path. In this example, the PDP chamber has dimensions of 3.4 inches in length and 0.06 inches ID for a volume of 0.00961 cubic inches or 157 μL. Similarly, the flow path volume throughout the pump block (for example, 31a through 31d) can also be provided with an ID of 0.06 inches. In an example, the length of the pump block polymer flow channel can be about 0.4 inches in length for a volume of 0.00113 cubic inches or 18.5 μL. Similarly, the polymer flow path from the polymer container 25 can be provided with an ID of 0.06 inches. In an example, the polymer supply line can be about 6.0 inches in length, for a volume of 0.01696 cubic inches or 278 μL. The inventor has found that providing the same ID throughout the polymer flow path (for example, interconnect tubing, joints, and connections) advantageously reduces the likelihood of air bubble formation in the polymer path due to cavitation effects. In addition, avoiding sharp corners in the polymer flow path reduces the amount of nucleation sites that could cause bubble formation in the polymer flow path.

In an exemplary embodiment, the interconnect tubing used for the polymer flow path is an amorphous sulfone polymer such as, for example, transparent Radel® tubing available from Solvay Advanced Polymers, LLC of Alpharetta, Ga. Radel® tubing has been found to be less likely to kink during installation and removal and because weekly maintenance can be optional. Radel® tubing also provides a low current density which provides less current fluctuation in the polymer path and reduced bubble formation. Transparent tubing, such as Radel® tubing, also allows visual confirmation of bubble removal from the polymer flow path. Radel® tubing can also provide a good seal for use with compression fittings. In an exemplary embodiment, 1.57 mm ID Radel® tube connection with fittings can be located between the polymer bottle 25 and check valve V1 in the upper (pump) block, and between the pump block 34 and the lower polymer block 15c. Other embodiments are possible. For example, PEEK™ tubing can be used. PEEK™ tubing is available from Victrex plc of Greenville, S.C. In embodiments using PEEK™ tubing, the PEEK™ tubing can have an ID that is smaller than the ID for Radel® tubing embodiments.

In an exemplary embodiment, the lower polymer block 15c can comprise a hydrophilic coating to minimize bubble formation. The polymer block can comprise large channels (for example, channels having an ID the same as the interconnect tubing) for polymer flow to provide a low current density to minimize bubble formation and heat generation. The pin valve PV solenoid can comprise a gap sufficiently large to provide the necessary sealing force. In an exemplary embodiment, the lower polymer block 15c can use KEL-F LiteTouch® fittings, available from Upchurch Scientific, Inc. of Oak Harbor, Wash., for sealed connection with the Radel® tubing. Lower polymer block 15c channels can comprise rounded elbows to reduce nucleation sites and unwanted bubbles. Further, the electrode length can extend, for example, about 3/16 inches beyond the lower polymer block 15c exit or overflow hole 48. The lower polymer block 15c internal bore can be treated with a hydrophilic coating material of, for example, approximately 1-2 μm in thickness. The bore can be configured to have minimal bubble nucleation sites. Further, the bore can be configured to comprise an ID of about 1.0 mm.

In an example, the polymer flow path through the compression fitting for the check valve V1 of the pump block 34 can comprise an ID that is the same ID as the channels within the pump block 34.

In an example, the buffer storage container 15 can comprise a jar having a volume of 67 millilLiters (mL). The jar can be tapered to prevent buffer spill. The jar can comprise a vent hole sufficiently large to not become easily plugged with buffer and to prevent the pin valve (PV) guide hole and vent hole from clogging. Dried polymer in the pin valve PV guide hole can result in sluggish action of the pin valve and lead to bubble formation near the pin valve tip due to constricted current path. A plugged PV guide hole can also lead to eventual arcing.

In an example, the PDP can be user replaceable. The PDP can also be factory reconditioned using reusable parts.

In an example, the PDP can be configured to generate pressure up to 1000 psi (70.3 kgf/cm²) for array filling. The force to generate 1000 psi can be controlled by limiting the current to the stepper motor, creating a stall condition. A service engineer at pump installation can set the pump current. The current value can be supplied by the pump supplier (for example, on a label attached to the front of the pump). Service can replace the appropriate values in the instrument calibration ini file. The entire pressurized polymer path can be configured to withstand a maximum 10 MegaPascal (MPa) (or, about 1450 psi).

Other embodiments of the present teachings will be apparent to those skilled in the art from consideration of the present specification and practice of the present teachings disclosed herein. It is intended that the present specification and examples be considered as exemplary only with a true scope and spirit of the teachings being indicated by the following claims and equivalents thereof.

All literature and similar materials cited in this application, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

While the teachings describes a multi-capillary electrophoresis instrument it is understood that the ideas extend, among other things, to a single capillary system or other electrophoresis approaches such as channel plates.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

What is claimed is:

1. A pump system comprising:
a first block;
an outlet opening formed in the first block;
a fluid chamber formed in the first block and in fluid communication with the outlet opening;
a buffer storage container connector adapted to retain a buffer storage container in fluid communication with the fluid chamber;
an electrode adjacent the buffer storage container connector and adapted to be in electrical communication with liquid in a buffer storage container when a buffer storage container is connected to the buffer storage container connector;
a polymer container connector adapted to form a fluid communication with polymer in a polymer container; and
a reciprocating piston pump in fluid communication with the fluid chamber and the polymer container connector.

2. The pump system of claim 1, further comprising a buffer jar connected to the buffer storage container connector, wherein the first block comprises a first fluid communication between the fluid chamber and the buffer jar.

3. The pump system of claim 1, further comprising a second fluid communication, wherein the polymer container connector comprises a check valve and the second fluid communication fluidly communicates the check valve and the pump.

4. The pump system of claim 3, wherein the check valve has a first inner diameter, and the second fluid communication has a second inner diameter that is the same size as the first inner diameter.

5. The pump system of claim 3, wherein the second fluid communication comprises a sidewall that exhibits a surface energy of about 30 dynes/centimeter or more.

6. The pump system of claim 3, wherein the second fluid communication comprises a hydrophilic sidewall.

7. The pump system of claim 1, wherein the reciprocating piston pump comprises a piston and a chamber, and the reciprocating piston pump is adapted to reciprocate the piston in the chamber.

8. The pump system of claim 7, wherein the piston comprises one or more of a gemstone material and a ceramic material.

9. The pump system of claim 7, wherein the piston comprises sapphire.

10. The pump system of claim 7, wherein the piston comprises a plasma-treated hydrophilic surface.

11. The pump system of claim 7, wherein the reciprocating piston pump further comprises a pump displacement motor adapted to reciprocate the piston.

12. The pump system of claim 7, wherein the first block further comprises a trap adapted to trap polymer adjacent the piston and prevent polymer from escaping from the first block.

13. The pump system of claim 1, further comprising a single capillary or multi-capillary array fluidly connected to the outlet opening.

14. The pump system of claim 13, wherein the outlet opening comprises an array port.

15. The pump system of claim 13, further comprising a connector comprising a threaded array knob and a double-tapered ferrule, fluidly connecting the array to the outlet opening.

16. The pump system of claim 1, wherein the polymer container connector comprises a vented cap.

17. The pump system of claim 1, wherein the polymer container connector comprises a vented cap and a tube, and the tube extends from the vented cap to the first block and is in fluid communication with the pump.

18. The pump system of claim 1, wherein the buffer storage container connector comprises a second block and a tube, and the tube provides a fluid communication between the first block and the second block.

19. The pump system of claim 1, wherein the first block comprises a block formed of a resin.

20. A capillary electrophoresis system comprising:
a pump system comprising a first block and an outlet opening formed in the first block, a fluid chamber formed in the first block and in fluid communication with the at least one outlet opening, a buffer storage container connector adapted to retain a buffer storage container in fluid communication with the fluid chamber, an electrode adjacent the buffer storage container connector and adapted to be in electrical communication with liquid in a buffer storage container when a buffer storage container is connected to the buffer storage container connector, a polymer container connector adapted to retain a polymer container, and a reciprocating piston pump in fluid communication with the fluid chamber and the polymer container connector;
a temperature regulated chamber comprising a container portion and an opening;
a single capillary or multi-capillary array disposed within the container portion and coupled to the outlet through the opening;
an excitation source; and
a detector assembly coupled to the single capillary or multi-capillary array.

21. The capillary electrophoresis system of claim 20, further comprising:
a buffer jar connected to the buffer storage container connector, wherein the first block comprises a first fluid communication between the fluid chamber and the buffer jar; and
a second fluid communication, wherein the polymer container connector comprises a check valve and the second fluid communication fluidly communicates the check valve and the pump.

22. The capillary electrophoresis system of claim 21, wherein the check valve has a first inner diameter, and the second fluid communication has a second inner diameter that is the same size as the first inner diameter.

23. The capillary electrophoresis system of claim 21, wherein the second fluid communication comprises a sidewall that exhibits a surface energy of about 30 dynes/centimeter or more.

24. The capillary electrophoresis system of claim 21, wherein the second fluid communication comprises a hydrophilic sidewall.

25. The capillary electrophoresis system of claim 21, wherein the reciprocating piston pump comprises a piston and a chamber, and the reciprocating piston pump is adapted to reciprocate the piston in the chamber.

26. The capillary electrophoresis system of claim 25, wherein the piston comprises a gemstone material.

27. The capillary electrophoresis system of claim 25, wherein the piston comprises sapphire.

28. The capillary electrophoresis system of claim 25, wherein the piston comprises a plasma-treated hydrophilic surface.

29. The capillary electrophoresis system of claim 25, wherein the reciprocating piston pump further comprises a pump displacement motor adapted to reciprocate the piston.

30. The capillary electrophoresis system of claim 25, wherein the first block further comprises a trap adapted to trap polymer adjacent the piston and prevent polymer from escaping from the first block.

31. The capillary electrophoresis system of claim 21, further comprising a single capillary or multi-capillary array fluidly connected to the outlet opening.

32. The capillary electrophoresis system of claim 31, wherein the outlet opening comprises an array port.

33. The capillary electrophoresis system of claim 32, further comprising a connector comprising a threaded array knob and a double-tapered ferrule.

34. The capillary electrophoresis system of claim 21, wherein the polymer container connector comprises a vented cap.

35. The capillary electrophoresis system of claim 21, wherein the polymer container connector comprises a vented cap and a tube, and the tube extends from the vented cap to the first block and is in fluid communication with the pump.

36. The capillary electrophoresis system of claim 21, wherein the buffer storage container connector comprises a second block and a tube, and the tube provides a fluid communication between the first block and the second block.

37. The capillary electrophoresis system of claim 21, wherein the first block comprises a block formed of a resin.

38. A method comprising:
reciprocating a pump piston in a first direction to draw fresh fluid into a chamber; and
reciprocating the pump piston in a second direction to cause the fresh fluid to exit the chamber and fill a single capillary or multi-capillary array of a capillary electrophoretic analyzer;
wherein the reciprocating comprises electrically controlling the pump piston movement.

39. The method of claim 38, further comprising drawing fresh fluid into the chamber once per every two or more fillings of the single capillary or multi-capillary array.

40. The method of claim 38, wherein drawing fresh fluid into the chamber comprises filling the chamber in a time period of from about 5 seconds to about 90 seconds.

41. The method of claim 40, wherein drawing fresh fluid into the chamber comprises filling the chamber in a time period of about 40 seconds or less.

42. The method of claim 38, wherein the pump piston reciprocates in a pump block comprising an outlet, and the pump block is attached to the single capillary or multi-capillary array with a high pressure seal for fluid communication between the outlet of the pump block and the single capillary or multi-capillary array.

43. The method of claim 38, wherein reciprocating the pump piston in the first direction decreases the pressure in the chamber, and wherein reciprocating the pump piston in the second direction increases the pressure in the chamber.

44. The method of claim 43, wherein electrically controlling the pump piston movement comprises controlling the speed of the piston to maintain a fluid pressure level.

45. The method of claim 44, further comprising:
monitoring the speed of the piston to detect a leak or bubble condition; and
stopping the piston movement upon detecting a leak or bubble condition.

46. The method of claim 38, wherein the fluid has a viscosity at least twice the viscosity of water.

47. The method of claim 46, wherein the fluid has a viscosity of from about 150 to about 550 times the viscosity of water.

48. The method of claim 46, wherein the fluid comprises a separation medium.

49. The method of claim 38, further comprising removing bubbles from the fluid.

50. The method of claim 38, further comprising washing at least one fluid path.

51. A method comprising:
reciprocating a pump piston in a first direction to cause fresh fluid to be received into a chamber; and
reciprocating the pump piston in a second direction to cause the fresh fluid to exit the chamber and enter a single capillary or multi-capillary array of a capillary electrophoretic analyzer;
wherein the reciprocating comprises controlling the pump piston movement using a programmable controller adapted to control a current to a stepper motor.

52. The method of claim 51, further comprising receiving fresh fluid into the chamber once per every two or more fillings of the single capillary or multi-capillary array.

53. The method of claim 51, wherein receiving fresh fluid into the chamber comprises filling the chamber in a time period of about 40 seconds or less.

54. The method of claim 51, further comprising:
monitoring the current to detect a leak or bubble condition; and
stopping the piston movement upon detecting the leak condition.
